(19) United States
(12) Patent Application Publication
(10) Pub. No.: US 2005/0004615 A1
(43) Pub. Date: Jan. 6, 2005

(54) Reconfigurable implantable cardiac monitoring and therapy delivery device

(76) Inventor: Richard S. Sanders, Stillwater, MN (US)

Correspondence Address: Crawford Maunu PLLC;Suite 390, 1270 Northland Drive, St. Paul, MN, 55120, US

(21) Appl. No.: 10/785,431

(22) Filed: Feb. 24, 2004

Related U.S. Application Data
(60) Provisional application No. 60/462,272, filed on Apr. 11, 2003.

(51) Int. Cl. A61N 1/375

(52) U.S. Cl. 607/36

(57) ABSTRACT

Methods and systems for implantable cardiac monitors reconfigurable to cardiac therapy devices. A device includes a housing with electrodes configured for cardiac activity sensing when the device is operated in monitoring mode, and sensing and energy delivery when operated in an energy delivery mode. A header may be configured to receive a cardiac lead, and may be associated with a switch to switch the device between monitoring and therapy modes in response to connecting one or more leads to the header. The device may include a transceiver that transmits the contents of the memory to a patient-external device. A method involves providing an implantable cardiac device configured to operate in a first mode as a loop recorder for monitoring cardiac activity and storing selected cardiac events, and operating in the second mode to monitor cardiac activity and provide cardiac stimulation therapy when the second mode is enabled.

### RELATED APPLICATIONS

This application claims the benefit of Provisional Patent Application Ser. No. 60/462,272, filed on Apr. 11, 2003, to which priority is claimed pursuant to 35 U.S.C. §119(e) and which is hereby incorporated herein by reference.

### FIELD OF THE INVENTION

The present invention relates generally to implantable medical devices and, more particularly, to methods and systems that provide for implantable cardiac monitors that are reconfigurable to cardiac therapy devices.

### BACKGROUND OF THE INVENTION

The healthy heart produces regular, synchronized contractions. Rhythmic contractions of the heart are normally controlled by the sinoatrial (SA) node, which is a group of specialized cells located in the upper right atrium. The SA node is the normal pacemaker of the heart, typically initiating 60-100 heartbeats per minute. When the SA node is pacing the heart normally, the heart is said to be in normal sinus rhythm.

If the heart's electrical activity becomes uncoordinated or irregular, the heart is denoted to be arrhythmic. Cardiac arrhythmia impairs cardiac efficiency and can be a potential life-threatening event. Cardiac arrhythmias have a number of etiological sources, including tissue damage due to myocardial infarction, infection, or degradation of the heart's ability to generate or synchronize the electrical impulses that coordinate contractions.

Bradycardia occurs when the heart rhythm is too slow. This condition may be caused, for example, by impaired function of the SA node, denoted sick sinus syndrome, or by delayed propagation or blockage of the electrical impulse between the atria and ventricles. Bradycardia produces a heart rate that is too slow to maintain adequate circulation.

When the heart rate is too rapid, the condition is denoted tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria may also adversely affect the ventricular rate.

Ventricular tachycardia occurs, for example, when electrical activity arises in the ventricular myocardium at a rate more rapid than the normal sinus rhythm. Ventricular tachycardia can quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating arrhythmias.

Typical Implantable cardioverter/defibrillators (ICDs) include one or more endocardial leads to which at least one defibrillation electrode is connected. Such ICDs are capable of delivering high-energy shocks to the heart, interrupting the ventricular tachyarrhythmia or ventricular fibrillation, and allowing the heart to resume normal sinus rhythm. ICDs may also include pacing functionality.

### SUMMARY OF THE INVENTION

The present invention is directed to methods and systems that provide for implantable cardiac monitors that are reconfigurable to cardiac therapy devices. In one embodiment of the present invention, an implantable cardiac device includes a housing with first and second electrodes coupled to the housing. The first and second electrodes may be configured for cardiac activity sensing when the device is operated in a monitoring mode. The cardiac device includes energy delivery circuitry coupled to the first and second electrodes, where the first and second electrodes may be configured for cardiac activity sensing and energy delivery when the device is operated in an energy delivery mode. A lead interface may be coupled to the housing and configured to receive a cardiac lead. A controller is coupled to the lead interface, recording circuitry, and energy delivery circuitry, the controller transitioning operation of the device from a monitoring mode (e.g. a loop-recording mode) to the energy delivery mode at least in part in response to coupling the cardiac lead to the lead interface.

The cardiac device may further include detection circuitry provided in the housing and coupled to the first and second electrodes, the detection circuitry configured to receive the cardiac signals. Memory may be provided in the housing and coupled to the detection circuitry, the memory configured to store selected cardiac signals. The cardiac device may include a programmable filter coupled to the detection circuitry, the programmable filter configurable in a first filtering mode for recording signals associated with the monitoring mode and configurable in a second filtering mode for cardiac event detection associated with the energy delivery mode. The cardiac device may further include a mode switch coupled to the controller, the mode switch configured to transition the cardiac device between the monitoring mode and the energy delivery mode.

The cardiac device may include a header used to connect leads to the device. The header may be associated with a switch to switch the device between monitoring and therapy modes in response to connecting one or more leads to the header. The cardiac device may be used with endocardial leads, epicardial leads, subcutaneous leads, and/or other leads or sensors.

The cardiac device may also include a transceiver that receives a transmit request signal and transmits the contents of the memory to a patient-external device in response to receipt of the transmit request signal. A receiver may be coupled to the controller, the controller switching the cardiac device between the monitoring (e.g. loop recording) mode and the energy delivery mode in response to the receiver receiving a switch request signal.

A system may include a patient-external device configured to send the transmit request signal to the transceiver of the cardiac device and receive the signals transmitted from the cardiac device. The patient-external device may include a patient actuatable trigger for triggering the recording and/or transmission of signals.

In another embodiment of the present invention, a cardiac monitoring and stimulation method involves providing an implantable cardiac device configured to operate in a first mode as a cardiac monitor, such as a loop recorder, for monitoring cardiac activity and storing selected cardiac events, and operating in a second mode to monitor cardiac activity and provide cardiac stimulation therapy when the second mode is enabled. Once enabled, the cardiac device operates in the second mode as a cardiac rhythm management system.

The method may further involve selecting cardiac events for storing via patient request. The device may continuously record cardiac events when operating in the first mode, storing selected cardiac events for subsequent analysis. The method may further involve transmitting the stored cardiac event data to a patient-external device.

The method may further involve connecting a lead to the cardiac device, switching the cardiac device between the first cardiac monitoring mode and the second monitoring and therapy mode. The therapy may involve defibrillation, cardioversion therapy, cardiac stimulation therapy, antitachycardia pacing therapy, and/or resynchronization pacing therapy, for example.

The method may further involve diagnosing a patient using the stored cardiac event data, to determine that the patient has a condition requiring use of a cardiac stimulation device. The device may then be configured to operate as a cardiac stimulation device. Switching the cardiac device between operating modes may be accomplished using a hardware switch, a software switch, a software upgrade or swap, or other switching approach.

### BRIEF DESCRIPTION OF THE DRAWINGS

FIG. 1 is a plan view of a reconfigurable cardiac device in accordance with the present invention, in its recording mode of operation;

FIG. 2 is a plan view of another embodiment of a reconfigurable cardiac device in accordance with the present invention, in its recording mode of operation;

FIG. 3 is a block diagram showing various components of a reconfigurable cardiac monitoring/stimulation device in accordance with an embodiment of the present invention;

FIG. 4 is a block diagram showing various components of a reconfigurable cardiac monitoring/stimulation device in accordance with an embodiment of the present invention;

FIG. 5 is a view of a reconfigurable cardiac device implanted in a patient in accordance with a cardiac therapy configuration of the present invention;

FIG. 6 is a view of a reconfigurable cardiac device implanted in a patient in accordance with another cardiac therapy configuration of the present invention;

FIG. 7 is a view of a dual-chamber reconfigurable cardiac device implanted in a patient's heart in accordance with an embodiment of the present invention in its therapeutic configuration; and

FIG. 8 is a view of a multi-chamber reconfigurable cardiac device implanted in a patient's heart in accordance with an embodiment of the present invention in its therapeutic configuration.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration, various embodiments in which the invention may be practiced. It is to be understood that other embodiments may be utilized, and structural and functional changes may be made without departing from the scope of the present invention.

An implantable cardiac device implemented in accordance with the principles of the present invention may include one or more of the features, structures, methods, or combinations thereof described below and in the above-identified Provisional Application. For example, a cardiac stimulator or monitor may be implemented to include one or more of the advantageous features and/or processes described below and in the above-identified Provisional Application. It is intended that such a stimulator, monitor or other implanted or partially implanted device need not include all of the features described herein, but may be implemented to include selected features that provide for unique structures and/or functionality.

One such device, termed an implantable reconfigurable cardiac device, is described herein to include various advantageous features and/or processes. It is understood that the description of features and processes within the context of a reconfigurable cardiac device in accordance with the present invention is provided for non-limiting illustrative purposes only, and that such features and processes may be implemented in other types of devices, including implantable and non-implantable devices. For example, features and processes described herein may be implemented in cardiac monitors, pacemakers, cardioverters/defibrillators, resynchronizers, and the like, including those devices disclosed in the various patents incorporated herein by reference. It is further understood that features and processes described herein may be implemented in devices that use one or more of transvenous, endocardial, epicardial, subcutaneous or surface electrodes, or devices that use combinations of these electrodes.

A reconfigurable cardiac monitoring/stimulation device may advantageously be used where it is desired to provide cardiac monitoring for diagnosis, before providing cardiac stimulation therapy. For example, a reconfigurable approach of the present invention allows upgrading the device from purely a monitoring and diagnostic system to a therapy delivery system for patients who develop or are diagnosed with conditions necessitating cardiac therapy. Exemplary devices and functionality that provide for such upgradeability are disclosed in commonly owned co-pending U.S. application Ser. No. 10/462,001, filed on Jun. 13, 2003, which is incorporated herein by reference. Structures and/or functionality implemented in accordance with the present invention may incorporate one or more features of the devices and methodologies disclosed in U.S. application Ser. No. 10/462,001.

A cardiac device in accordance with the present invention may implement functionality traditionally provided by cardiac monitors as are known in the art, and after reconfiguration, provide cardiac therapy. Exemplary cardiac monitoring circuitry, structures and functionality, aspects of which may be incorporated in a cardiac device in accordance with the present invention of a type contemplated herein, are disclosed in commonly owned U.S. Pat. Nos. 5,313,953; 5,388,578; and 5,411,031, which are hereby incorporated herein by reference in their respective entireties.

A reconfigurable cardiac monitoring/stimulation device may be implanted under the skin in the chest region of a patient. The cardiac device may, for example, be implanted subcutaneously, positioned on the patient's front, back, side, or other body locations suitable for sensing cardiac activity and/or delivering cardiac stimulation therapy. It is understood that elements of the cardiac device in its therapeutic configuration may be located at several different body locations, such as in the chest, abdominal, or subclavian region, with electrode elements respectively positioned at different regions near, around, in, or on the heart. For example, intrathoracic lead/electrode elements of the cardiac device may be positioned on or within the heart, great vessel or coronary vasculature.

The primary housing (e.g., the active or non-active can) of the cardiac device, for example, may be configured for positioning outside of the rib cage at an intercostal or subcostal location, within the abdomen, or in the upper chest region (e.g., subclavian location, such as above the third rib). A transthoracic configuration of the cardiac device in its therapeutic configuration typically employs one or more electrodes located on, or extending from, the primary housing and/or at other locations about, but not in direct contact with, the heart, great vessel or coronary vasculature. Such electrodes are generally referred to herein as subcutaneous electrodes, it being understood that surface electrodes may also be employed in certain configurations. One or more subcutaneous electrode arrays, for example, may be used to sense cardiac activity and deliver cardiac stimulation energy in a cardiac device in accordance with the therapeutic configuration of the present invention employing an active can or non-active can. Electrodes can be situated at anterior and/or posterior locations relative to the heart.

A cardiac device typically includes a controller or control system that can alter the configuration and operating modes of the device. For example, the controller can configure the cardiac device to operate in a first mode for cardiac monitoring and recording of cardiac events, and to operate in a therapy configuration using cardiac monitoring and stimulation circuitry. Alterations in the operating configuration or mode of a reconfigurable cardiac device in accordance with the present invention may be initiated and controlled in a variety of ways. For example, the cardiac device may switch operating modes or configurations in response to a configuration signal received from a patient-external signal source, such as from a programmer or patient/clinician controlled activator. The controller of a cardiac device may also change modes or configurations in response to a predetermined condition, such as when a lead system is connected to a header of the device, for example.

In particular configurations, systems and methods may perform functions traditionally performed by pacemakers, such as providing various pacing therapies as are known in the art. Exemplary pacemaker circuitry, structures and functionality, aspects of which may be incorporated in a cardiac device in accordance with the present invention of a type contemplated herein, are disclosed in commonly owned U.S. Pat. Nos. 4,562,841; 5,284,136; 5,376,106; 5,036,849; 5,540,727; 5,836,987; 6,044,298; and 6,055,454, which are hereby incorporated herein by reference in their respective entireties.

Certain system configurations illustrated herein are generally described as capable of implementing various functions traditionally performed by an implantable cardioverter/defibrillator (ICD), and may operate in numerous cardioversion/defibrillation modes as are known in the art. Exemplary ICD circuitry, structures and functionality, aspects of which may be incorporated in a cardiac device in accordance with the present invention, are disclosed in commonly owned U.S. Pat. Nos. 5,133,353; 5,179,945; 5,314,459; 5,318,597; 5,620,466; and 5,662,688, which are hereby incorporated herein by reference in their respective entireties.

It is also understood that the components and functionality depicted in the figures and described herein may be implemented in hardware, software, or a combination of hardware and software. It is further understood that the components and functionality depicted as separate or discrete blocks/elements in the figures may be implemented in combination with other components and functionality, and that the depiction of such components and functionality in individual or integral form is for purposes of clarity of explanation, and not of limitation.

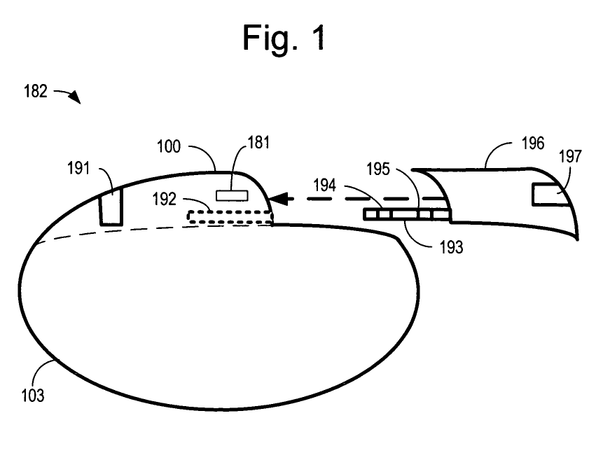

FIG. 1 is a plan view of a reconfigurable cardiac device 182 in accordance with the present invention, in its recording mode of operation. A can 103 is illustrated incorporating a header 100 for removable attachment of an electrode module 196. The header 100 includes a female coupler 192 configured to accept a male coupler 193 from the electrode module 196. The male coupler 193 is shown having two electrode contacts 193, 194 for coupling one or more electrodes 197 through the electrode module 196 to the can 103. An electrode 191 is illustrated on the header 100 of the can 103. A proximity switch 181 is shown in the header 100, which may be used to recognize the attachment of the electrode module 196 to the can 103. The proximity switch 181 may also be useful for switching the cardiac device 182 from its recording mode to its therapy mode, as will be further described later.

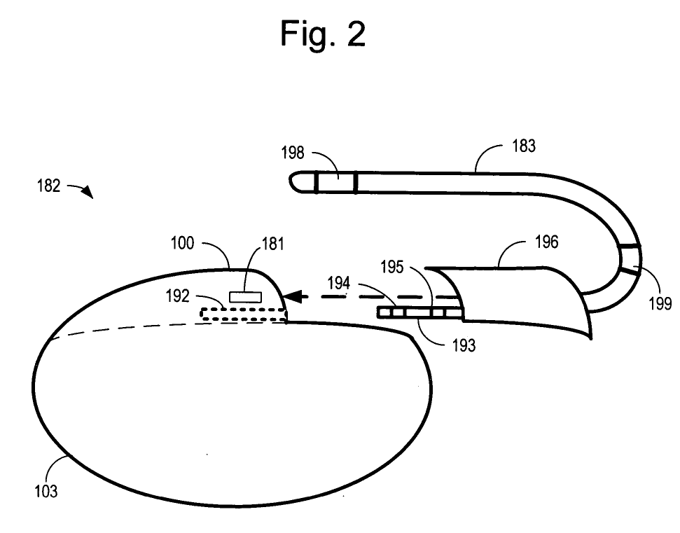

FIG. 2 is a plan view of another embodiment of a reconfigurable cardiac device 182 in accordance with the present invention, in its recording mode of operation. In the embodiment illustrated in FIG. 2, a first recording electrode 198 and a second recording electrode 199 are attached to the can 103 through the header 100, using the electrode module 196. The first recording electrode 198 and the second recording electrode 199 may be located on a lead 183, or may be located directly in or on the electrode module 196. The reconfigurable cardiac device 182 may be initially implanted in a patient, and used for recording cardiac events helpful for diagnosis or verification of diagnosis. Subsequent to diagnosis or verification of diagnosis, the cardiac device may then be reconfigured for cardiac therapy.

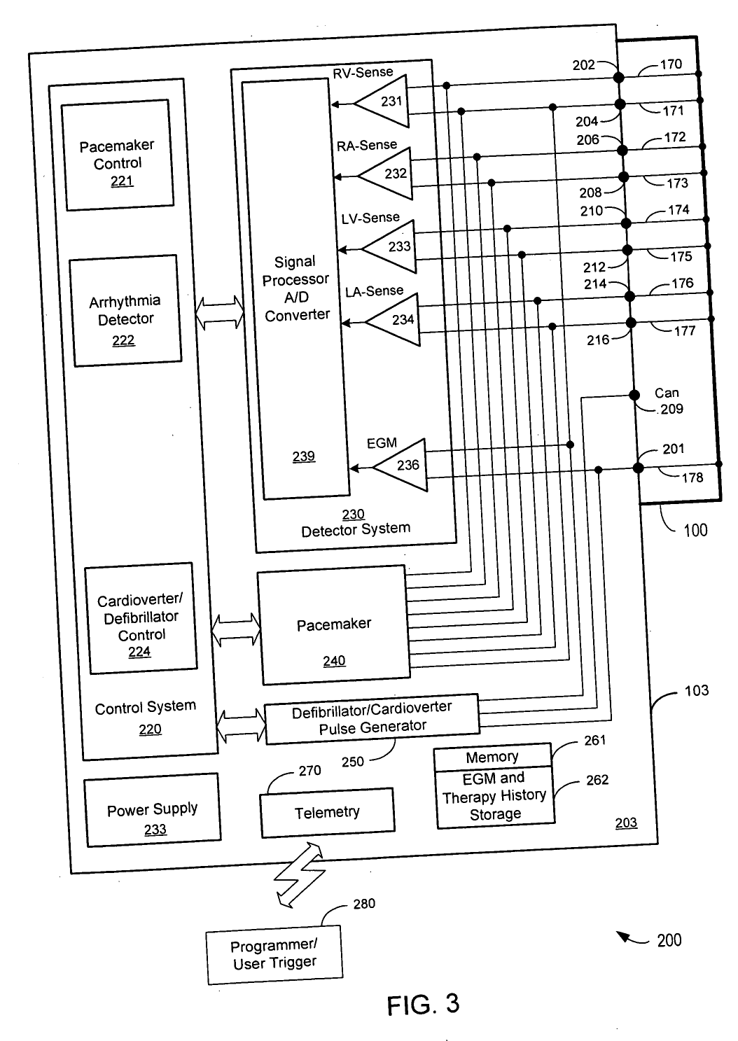

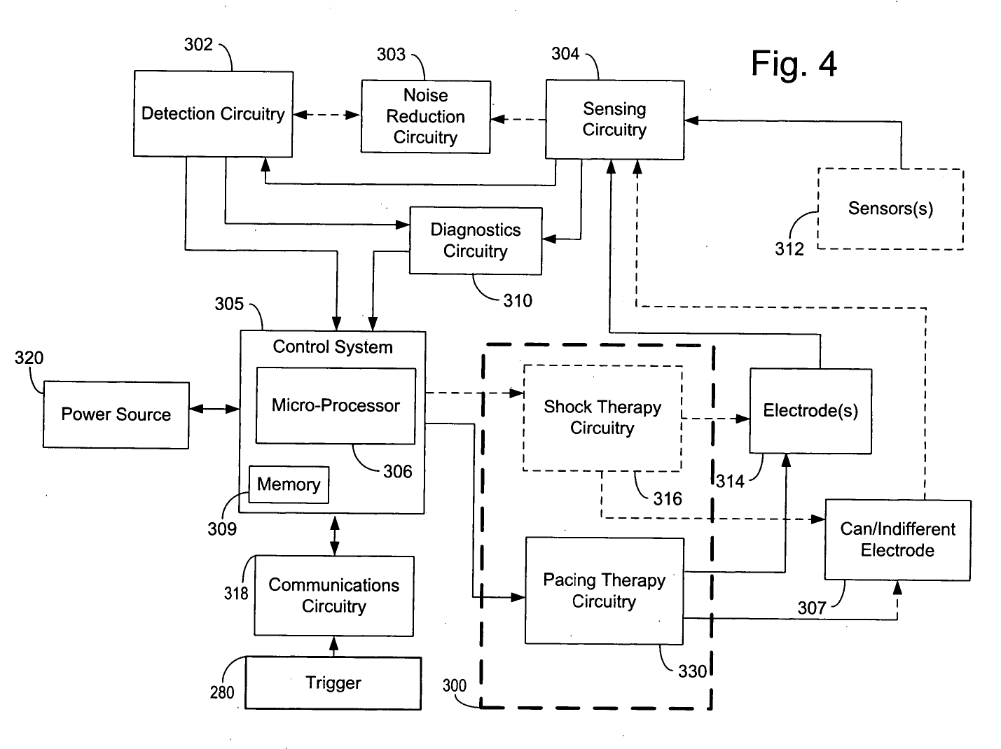

FIGS. 3 and 4 illustrate embodiments of the reconfigurable cardiac device common to both modes of operation, with selected elements switchable or enableable depending on which mode of operation is desired. For example, the device 182 (FIGS. 1 and 2) may operate in a recording mode, loop recording EGM signals and storing signals of interest, when the proximity switch 181 indicates a electrode module 196 is attached to the device 182. The device may switch to a therapy mode of operation in response to removal of the electrode module 196 and attachment of a cardiac therapy lead, such as will be described below.

FIG. 3 illustrates an intrathoracic reconfigurable cardiac device according to the present invention. FIG. 4 illustrates a transthoracic reconfigurable cardiac device according to another embodiment of the present invention. Although a cardiac device in accordance with the present invention may incorporate components and functionality provided by one or both of intrathoracic and transthoracic elements, such components and functionality are presented in separate figures for purposes of simplicity and clarity.

Moreover, it is understood that the embodiments depicted in FIGS. 3 and 4 may share similar components, and that such components may be implemented using a common component or implemented as separate components. Further, the embodiments depicted in FIGS. 3 and 4 may share similar functions. For example, the circuitry shown in FIG. 3 includes a control system 220, which may be the same or different system as that shown as a control system 305 in FIG. 4.

The system 200 shown in FIG. 3 is suitable for implanting in a patient and used for recording cardiac related signals in a first operating mode. In the first operating mode, the system 200 may include only sensors in or on a housing 103 or the system 200 may include one or more other sensors and/or electrodes as will be further described below. When the system 200 is configured to operate in a second monitoring and therapy mode, typically cardiac electrodes are attached to the housing 103, such as, for example, using the header 100. For purposes of illustration, the intrathoracic system 200 depicted in FIG. 3 will be described as having CFM functionality. It is understood that the systems shown in FIGS. 1-8 may be configured to perform conventional pacemaker and/or cardioversion/defibrillator functions in addition to, or to the exclusion of, CFM functions. The system 200 shown in FIG. 3 is divided into functional blocks. There exist many possible configurations in which these functional blocks can be arranged. The configuration depicted in FIG. 3 is one possible functional arrangement.

Conductors 102 and 104 are available in the header 100 for connecting and transmitting sense and pacing signals between terminals 202 and 204 of the cardiac device and right ventricular (RV)-tip and RV-coil electrodes, respectively. Conductor 101 is available in the header 100 for connecting and transmitting signals between the SVC coil and terminal 201 of the cardiac device. Conductor 106 is available in the header 100 for connecting and transmitting signals between the right-atrial (RA)-tip electrode and terminal 206 and conductor 108 is available in the header 100 for connecting and transmitting signals between the RA-ring electrode and terminal 208.

Conductors 110, 112 are available in the header 100 for connecting and transmitting sense and pacing signals between terminals 210, 212 of the cardiac device and left-ventricular (LV)-tip and LV-ring electrodes respectively. Conductor 114 is available in the header 100 for connecting and transmitting signals between the left-atrial (LA)-tip electrode and terminal 214 and conductor 116 is available in the header 100 for connecting and transmitting signals between the LA-ring electrode and terminal 216. A can electrode 209 coupled to a housing 103 of the cardiac device is also provided.

The device circuitry 203 is encased in the hermetically sealed housing 103 suitable for implanting in a human body. Power to the cardiac device 200 is supplied by an energy source 233, such as an electrochemical battery, fuel cell, or external energy source, housed within, or otherwise supplying energy to, the device 200. In one embodiment, the reconfigurable circuitry 203 is a programmable microprocessor-based system, including a control system 220, detector system 230, pacemaker 240, cardioverter/defibrillator pulse generator 250 and a memory circuit 261.

The memory circuit 261 stores parameters for various pacing, defibrillation, and sensing modes and stores data indicative of cardiac signals and signals from other sensors received by other components of the device circuitry 203. Memory is provided for storing historical EGM signals 262, which may be used on-board for various purposes and transmitted to an external programmer unit/trigger 280 or other patient-external device as required. The memory circuit 261 may be utilized in a loop recording mode, continuously recording data from electrodes and/or sensors until a cardiac event occurs, and then storing in long-term memory all or part of the recorded sensor/electrode information preceding, during, and after the cardiac event. The memory and signal storage may also be triggered by a patient or user, actuatable by the external programmer unit/trigger 280.

The control system 220 may use various control subsystems including pacemaker control 221, cardioverter/defibrillator control 224, and arrhythmia detector 222. The control system 220 may encompass additional functional components (not shown) for controlling the device circuitry 203. The control system 220 and memory circuit 261 cooperate with other components of the device circuitry 203 to perform operations involving synchronized pacing, in addition to other sensing, pacing and defibrillation functions.

Telemetry circuitry 270 is additionally coupled to the device circuitry 203 to allow the cardiac device 200 to communicate with the external programmer unit/trigger 280. In one embodiment, the telemetry circuitry 270 and the programmer unit/trigger 280 use a wire loop antenna and a radio frequency telemetric link to receive and transmit signals and data between the programmer unit/trigger 280 and telemetry circuitry 270. In this manner, programming commands may be transferred to the device circuitry 203 from the programmer unit/trigger 280 during and after implant. In addition, stored cardiac data, along with other data, may be transferred to the programmer unit/trigger 280 from the cardiac device 200, for example.

Cardiac signals sensed through use of the RV-tip and LV-tip electrodes are near-field signals, as are known in the art. More particularly, a signal derived from the right ventricle is detected as a voltage developed between the RV-tip electrode and the RV-coil. RV-tip and RV-coil electrodes are shown coupled to an RV-sense amplifier 231 located within the detector system 230. Signals received by the RV-sense amplifier 231 are communicated to the signal processor and A/D converter 239. The RV-sense amplifier 231 serves to sense and amplify the signals. The signal processor and A/D converter 239 convert the R-wave signals from analog to digital form and communicate the signals to the control system 220.

Signals derived from the left ventricle are detected as a voltage developed between the LV-tip electrode and the LV-ring electrode. LV-tip and LV-ring electrodes are shown coupled to an LV-sense amplifier 233 located within the detector system 230. Signals received by the 233 are communicated to the signal processor and A/D converter 239. The LV-sense amplifier 233 serves to sense and amplify the signals. The signal processor and A/D converter 239 convert the R-wave signals from analog to digital form and communicate the signals to the control system 220.

Cardiac signals sensed through use of one or both of the RV-coil and the SVC-coil are far-field signals, also referred to as morphology or shock channel signals, as are known in the art.. More particularly, a shock channel signal is detected as a voltage developed between the RV-coil and the SVC-coil. A shock channel signal may also be detected as a voltage developed between the RV-coil and the SVC-coil coupled to the can electrode 209. Shock channel signals developed using appropriate combinations of the RV-coil, SVC-coil, and can electrode are sensed and amplified by a shock EGM amplifier 236 located in the detector system 230. The output of the EGM amplifier 236 is coupled to the control system 220 via the signal processor and A/D converter 239.

RA-tip and RA-ring electrodes are shown coupled to an RA-sense amplifier 232 located within the detector system 230. Atrial sense signals received by the RA-sense amplifier 232 in the detector system 230 are communicated to an A/D converter 239. The RA-sense amplifier serves to sense and amplify the A-wave signals of the right atrium. The A/D converter 239 converts the sensed signals from analog to digital form and communicates the signals to the control system 220.

A-wave signals originating in the left atrium are sensed by the LA-tip and LA-ring electrodes. The A-waves are sensed and amplified by the LA-sense amplifier 234 located in the detector system. The LA-sense amplifier serves to sense and amplify the A-wave signals of the left atrium. The A/D converter 239 converts the sensed signals from analog to digital form and communicates the signals to the control system 220.

The pacemaker 240 communicates pacing signals to the pacing electrodes, RV-tip, RA-tip, LV-tip and LA-tip, according to a pre-established pacing regimen under appropriate conditions. Blanking circuitry (not shown) is employed in a known manner when ventricular or atrial pacing pulses are delivered, such that the ventricular channels, atrial channels, and shock channel are properly blanked at the appropriate time and for the appropriate duration.

A reconfigurable cardiac device that incorporates CFM functionality may be configured in its therapy operating mode to improve pumping function by altering contraction sequences in a manner distinct from conventional bradycardia pacing. To treat bradycardia, for example, pacing may be performed when the heart rate is not fast enough or the atrioventricular (AV) interval is too long.

To improve pumping function, two or more heart chambers may be paced simultaneously or in phased sequence, thus coordinating inefficient or non-existent contraction sequences. For example, a pacing mode may be employed to pace both the left ventricle, LVP, and the right ventricle, RVP, after a sensed atrial contraction, AS. Such a pacing mode may mitigate pathological ventricular conduction delays, thereby improving the pumping function of the heart.

FIG. 4 illustrates a transthoracic reconfigurable cardiac device according to another embodiment of the present invention. According to the configuration shown in FIG. 4, a cardiac device incorporates a processor-based control system 305 which includes a micro-processor 306 coupled to appropriate memory 309, it being understood that any logic-based control architecture may be used. The control system 305 is coupled to circuitry and components to sense, detect, and analyze electrical signals produced by the heart and record selected signals in the memory 309. The memory 309 may be utilized in a loop recording mode, continuously recording data from electrodes and/or sensors until a cardiac event occurs, and then storing in long-term memory all or part of the recorded sensor/electrode information preceding, during, and after the cardiac event. When configured in a monitoring and stimulation mode, the control system 305 may prompt delivery of electrical stimulation energy to the heart under predetermined conditions to treat cardiac arrhythmias. In certain configurations, the control system 305 and associated components may also provide pacing therapy to the heart. The electrical energy delivered by the cardiac device may be in the form of low energy pacing pulses or high energy pulses for cardioversion or defibrillation.

Cardiac signals are sensed, for example, using the subcutaneous electrode(s) 314 and the can or indifferent electrode 307 provided on the cardiac device housing. Cardiac signals may also be sensed using only the subcutaneous electrodes 314, such as in a non-active can configuration. Cardiac signals may also be sensed using only sensors and/or electrodes in or on the can when the control system 305 is operating in the first (monitoring and recording) mode. As such, unipolar, bipolar, or combined unipolar/bipolar electrode configurations may be employed. The sensed cardiac signals are received by sensing circuitry 304, which includes sense amplification circuitry and may also include filtering circuitry and an analog-to-digital (A/D) converter. The sensed cardiac signals processed by the sensing circuitry 304 may be received by noise reduction circuitry 303, which may further reduce noise before signals are sent to the detection circuitry 302. Noise reduction circuitry 303 may also be incorporated after detection circuitry 302 in cases where high power or computationally intensive noise reduction algorithms are required.

In the illustrative configuration shown in FIG. 4, the detection circuitry 302 is coupled to, or otherwise incorporates, noise reduction circuitry 303. The noise reduction circuitry 303 operates to improve the signal-to-noise ratio of sensed cardiac signals by removing noise content of the sensed cardiac signals introduced from various sources. Typical types of transthoracic cardiac signal noise includes electrical noise and noise produced from skeletal muscles, for example.

Detection circuitry 302 typically includes a signal processor that coordinates analysis of the sensed cardiac signals and/or other sensor inputs to detect cardiac arrhythmias, such as, in particular, tachyarrhythmia. Rate-based (e.g., rate zone-based), pattern and rate-based, and/or morphological discrimination algorithms may be implemented by the signal processor of the detection circuitry 302 to detect and verify the presence and severity of an arrhythmic episode.

Exemplary arrhythmia detection and discrimination circuitry, structures, and techniques, aspects of which may be implemented by a cardiac device in accordance with the present invention of a type contemplated herein, are disclosed in commonly owned U.S. Pat. Nos. 5,301,677 and 6,438,410, which are hereby incorporated herein by reference in their respective entireties. Exemplary pattern and rate-based arrhythmia detection and discrimination circuitry, structures, and techniques, aspects of which may be implemented by a cardiac device in accordance with the present invention of a type contemplated herein, are disclosed in U.S. Pat. Nos. 6,487,443; 6,259,947; 6,141,581; 5,855,593; and 5,545,186, which are hereby incorporated herein by reference in their respective entireties. Arrhythmia detection methodologies particularly well suited for implementation in subcutaneous cardiac stimulation systems are described in further detail in the above-identified provisional application.

The detection circuitry 302 communicates cardiac signal information to the control system 305. Memory circuitry 309 of the control system 305 contains parameters for operating in various sensing, defibrillation, and pacing modes, and stores data indicative of cardiac signals received by the detection circuitry 302. The memory circuitry 309 may also be configured to store historical ECG and therapy data, which may be used for various purposes and transmitted to an external receiving device as needed or desired.

In certain configurations, the cardiac device may include diagnostics circuitry 310. The diagnostics circuitry 310 typically receives input signals from the detection circuitry 302 and the sensing circuitry 304. The diagnostics circuitry 310 provides diagnostics data to the control system 305, it being understood that the control system 305 may incorporate all or part of the diagnostics circuitry 310 or its functionality. The control system 305 may store and use information provided by the diagnostics circuitry 310 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to and after therapy delivery.

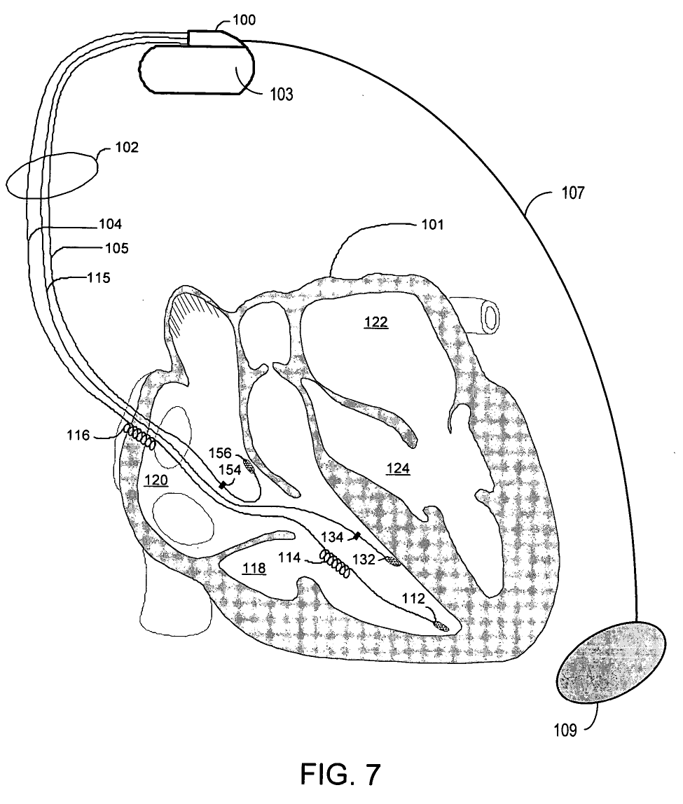

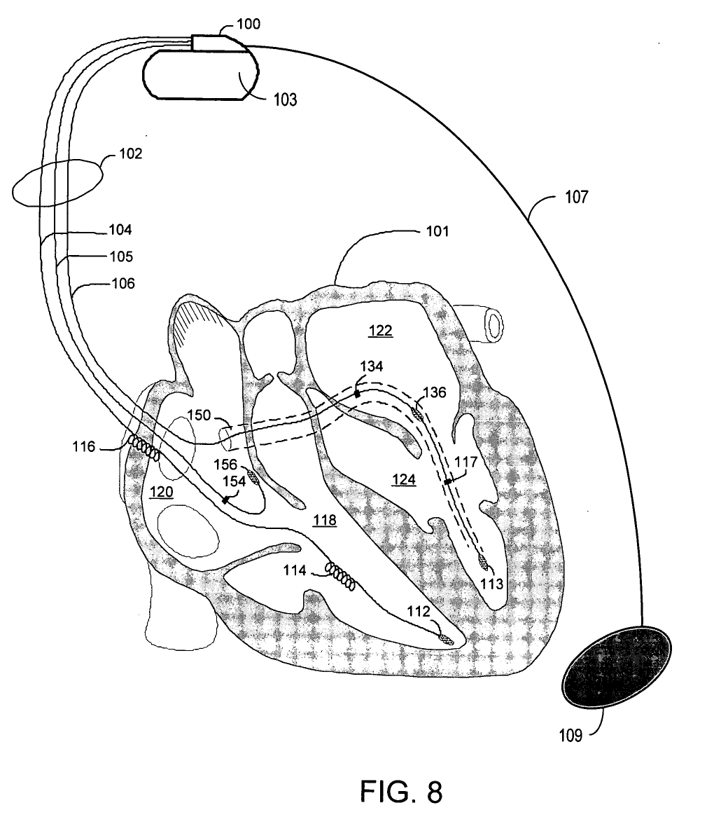

A reconfigurable cardiac device in accordance with the present invention includes a therapy portion 300, which is disabled when the cardiac device is operated in a first monitoring and recording mode, and enabled when operating in a second monitoring and therapy mode. The therapy portion 300 may be physically switchable, using a hardware switch, to enable/disable the therapy portion 300. The therapy portion may be enabled/disabled via control signals from the control system 305. It is also contemplated that a combination of hardware and software may be used to enable/disable the therapy portion 300. For example, the header 100 (see for example, FIGS. 7 and 8) may include a proximity switch or other component required to enable the therapy portion 300. The control system 305 may require detection of one or more therapy electrodes before enabling the therapy portion 300.

According to a configuration that provides transthoracic cardioversion and defibrillation therapies, the control system 305 processes cardiac signal data received from the detection circuitry 302 and initiates appropriate tachyarrhythmia therapies to terminate cardiac arrhythmic episodes and return the heart to normal sinus rhythm. The control system 305 is coupled to shock therapy circuitry 316. The shock therapy circuitry 316 is coupled to the subcutaneous electrode(s) 314 and the can or indifferent electrode 307 of the cardiac device housing. Upon command, the shock therapy circuitry 316 delivers cardioversion and defibrillation stimulation energy to the heart in accordance with a selected cardioversion or defibrillation therapy. In a less sophisticated configuration, the shock therapy circuitry 316 is controlled to deliver defibrillation therapies, in contrast to a configuration that provides for delivery of both cardioversion and defibrillation therapies. Exemplary ICD high energy delivery circuitry, structures and functionality, aspects of which may be incorporated in a cardiac device in accordance with the present invention of a type contemplated herein, are disclosed in commonly owned U.S. Pat. Nos. 5,372,606; 5,411,525; 5,468,254; and 5,634,938, which are hereby incorporated herein by reference in their respective entireties.

In accordance with another configuration, the transthoracic system of a cardiac device in accordance with the present invention incorporates a cardiac pacing capability. As is shown in FIG. 4, the cardiac device includes pacing therapy circuitry 330 that is coupled to the control system 305 and the subcutaneous and can/indifferent electrodes 314, 307. Upon command, the pacing therapy circuitry delivers pacing pulses to the heart in accordance with a selected pacing therapy. Control signals, developed in accordance with a pacing regimen by pacemaker circuitry within the control system 305, are initiated and transmitted to the pacing therapy circuitry 330 where pacing pulses are generated. A pacing regimen may be modified by the control system 305.

A number of cardiac pacing therapies may be delivered via the pacing therapy circuitry 330 as shown in FIG. 4. Alternatively, cardiac pacing therapies may be delivered via the shock therapy circuitry 316, which effectively obviates the need for separate pacemaker circuitry. Examples of various approaches for delivering cardiac pacing therapies via the shock therapy circuitry 316 are disclosed in commonly owned U.S. patent application Ser. No. 10/377,274, filed Feb. 28, 2003, which is hereby incorporated herein by reference.

The cardiac device shown in FIG. 4 may be configured to receive signals from one or more physiologic and/or non-physiologic sensors 312. Depending on the type of sensor employed, signals generated by the sensors 312 may be communicated to transducer circuitry coupled directly to the detection circuitry or indirectly via the sensing circuitry. It is noted that certain sensors can transmit sense data to the control system 305 without processing by the detection circuitry 302.

Communications circuitry 318 is coupled to the micro-processor 306 of the control system 305. The communications circuitry 318 allows the cardiac device to communicate with one or more receiving devices or systems situated external to the cardiac device. By way of example, the cardiac device may communicate with a patient-worn, portable or bed-side communication system or patient actuatable trigger via the communications circuitry 318. In one configuration, one or more physiologic or non-physiologic sensors (subcutaneous, cutaneous, or external of patient) may be equipped with a short-range wireless communication interface, such as an interface conforming to a known communications standard, such as Bluetooth or IEEE 802 standards. Data acquired by such sensors may be communicated to the cardiac device via the communications circuitry 318. It is noted that physiologic or non-physiologic sensors equipped with wireless transmitters or transceivers may communicate with a receiving system external of the patient.

The communications circuitry 318 may allow the cardiac device to communicate with an external programmer/trigger 280. In one configuration, the communications circuitry 318 and the programmer/trigger 280 use a wire loop antenna and a radio frequency telemetric link, as is known in the art, to receive and transmit signals and data between the programmer unit and communications circuitry 318. In a manner similar to that described above with regard to the intrathoracic system block diagram of FIG. 3, programming commands and data may be transferred between the cardiac device and the programmer/trigger 280 during and after implant. Using a programmer, a physician is able to set or modify various parameters used by the cardiac device. For example, a physician may set or modify parameters affecting sensing, detection, pacing, and defibrillation functions of the cardiac device, including pacing and cardioversion/defibrillation therapy modes.

Power to the cardiac device is supplied by a power source 320 disposed within a hermetically sealed housing of the cardiac device. The power source 320 may be the same (or a different) source of power as the power source 233 shown in FIG. 3. In one configuration, the power source 320 includes a rechargeable battery. According to this configuration, charging circuitry is coupled to the power source 320 to facilitate repeated non-invasive charging of the power source 320. The communications circuitry 318, or separate receiver circuitry, is configured to receive RF energy transmitted by an external RF energy transmitter. The cardiac device may, in addition to a rechargeable power source, include a non-rechargeable battery. It is understood that a rechargeable power source need not be used, in which case a long-life non-rechargeable battery is employed.

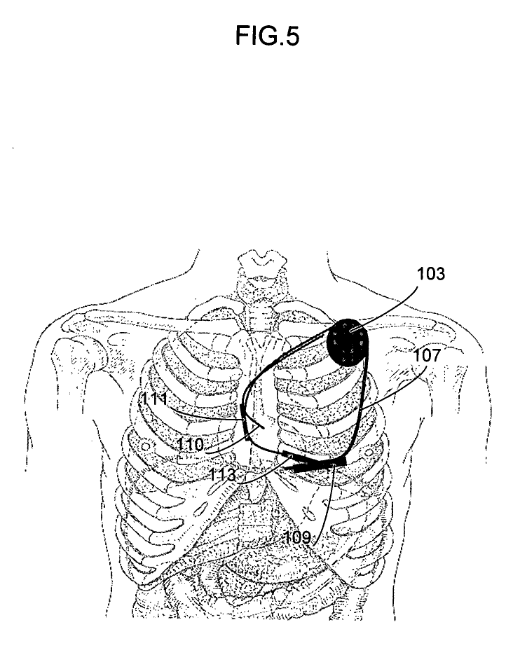

FIGS. 5-8 illustrate embodiments of the present invention after configuring the cardiac device from its first monitoring mode, to its second therapy mode. Referring now to FIG. 5 of the drawings, there is shown a reconfigurable cardiac device, in its therapy configuration, implanted in the chest region of a patient in accordance with an embodiment of the present invention. A typical cardiac device in accordance with the cardiac monitoring and stimulation mode of present invention may include one or more subcutaneous electrodes and/or one or more transvenous, epicardial, and/or endocardial electrodes. With regard to the particular configuration shown in FIG. 5, the cardiac device includes a housing 103 within which various cardiac sensing, detection, processing, and energy delivery circuitry may be housed. Communications circuitry is disposed within the housing 103 for facilitating communication between the cardiac device and an external communication device, such as a patient actuatable trigger, portable or bed-side communication station, patient-carried/worn communication station, or external programmer, for example. The communications circuitry may also facilitate unidirectional or bidirectional communication with one or more external, cutaneous, or subcutaneous physiologic or non-physiologic sensors.

An electrode support assembly defines a physically separable unit relative to the housing 103. The electrode support assembly includes mechanical and electrical couplings that facilitate mating engagement with corresponding mechanical and electrical couplings of the housing 103. For example, a header block arrangement may be configured to include both electrical and mechanical couplings that provide for mechanical and electrical connections between the rigid electrode support assembly and housing 103. Alternatively, a mechanical/electrical coupler may be used to establish mechanical and electrical connections between the electrode support assembly and housing 103. In such a configuration, a variety of different electrode support assemblies of varying shapes, sizes, and electrode configurations may be made available for physically and electrically connecting to a reconfigurable cardiac device in accordance with the present invention.

In the configuration shown in FIG. 5, a subcutaneous electrode 109 can be positioned under the skin in the chest region and situated distal from the housing 103. The subcutaneous and, if applicable, housing electrode(s) may be positioned about the heart at various locations and orientations, such as at various anterior and/or posterior locations relative to the heart. The subcutaneous electrode 109 is electrically coupled to circuitry within the housing 103 via a lead assembly 107. One or more conductors (e.g., coils or cables) are provided within the lead assembly 107 and electrically couple the subcutaneous electrode 109 with circuitry in the housing 103. One or more sense, sense/pace or defibrillation electrodes may be situated on the elongated structure of the electrode support, the housing 103, and/or the distal electrode assembly.

The cardiac device shown in FIG. 5 further includes an endocardial lead system, which is electrically coupled to circuitry within the housing 103 via one or more transvenous leads. The endocardial lead system may be implanted using a conventional transvenous lead delivery procedure. The endocardial lead system may include a single lead for implant within or to a single heart chamber (atrial or ventricular chamber) or multiple heart chambers (e.g., single pass lead). More than one lead may be deployed (e.g., right and/or left heart leads) for implant within one or multiple heart chambers (e.g., multisite or multi-chamber configuration). As such, a cardiac device in accordance with the present invention may be implanted to provide intrathoracic sensing and/or stimulation therapy in one, two, three, or four heart chambers.

In FIG. 5, an atrial lead system includes a lead (e.g., right atrial lead) for electrically coupling the housing circuitry with one or more atrial electrodes 110. A ventricular defibrillation lead system may include one or two leads for electrically coupling the housing circuitry with one or more ventricular electrodes. The ventricular defibrillation lead system may include, for example, a right ventricular electrode 113 and an electrode 111 positioned in the superior vena cava.

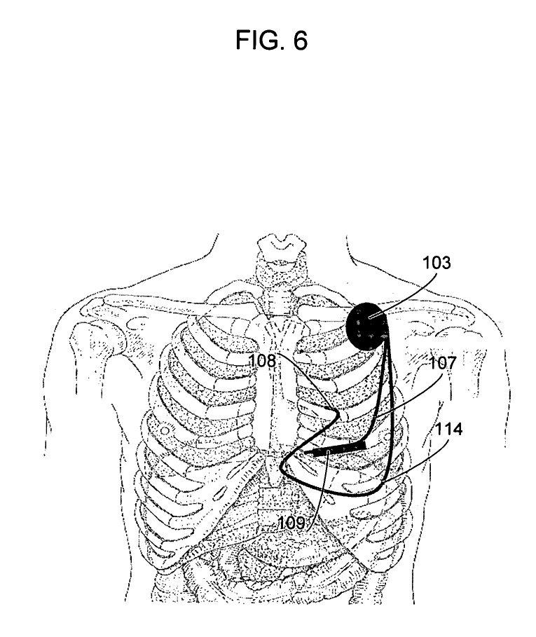

The cardiac device shown in FIG. 6 includes the subcutaneous electrode and housing components shown in FIG. 5, but-employs one or more epicardial or transvenous lead systems instead of the endocardial lead approach-shown in FIG. 5. A typical transvenous lead system may include one or more electrodes adapted for implant within a great vessel (e.g., coronary or pulmonary vessel) or coronary vasculature. A typical epicardial lead system may include one or more patch-type and/or screw-in electrodes or other electrode configuration that contacts the epicardium of the heart.

In FIG. 6, an intrathoracic lead 114 includes one or more distal electrodes 108 that may be configured for epicardial or transvenous cardiac activity sensing and/or stimulation energy delivery. As shown, a single lead 114 electrically couples the intrathoracic electrode(s) 108 with circuitry provided in the housing 103. It is appreciated that one or more intrathoracic leads 114 may be deployed to provide sensing and stimulation energy delivery for one or more chambers of the heart.

FIG. 7 shows one embodiment of a cardiac device in accordance with the present invention in its cardiac stimulation mode, useful for synchronized multisite sensing or pacing within a heart chamber. The cardiac device includes a housing 103 electrically and physically coupled to an intracardiac lead system 102 through a header 100. The intracardiac lead system 102 includes one or more electrodes used for pacing, sensing, or defibrillation. In the particular embodiment shown in FIG. 7, the intracardiac lead system 102 includes first and second right ventricular lead systems 104, 115 and a right atrial lead system 105. In one embodiment, the right ventricular lead system 104 is configured as an integrated bipolar pace/shock lead.

The first right ventricular lead system 104 includes an SVC-coil 116, an RV-coil 114, and an RV-tip electrode 112. The RV-coil 114, which may alternatively be configured as an RV-ring electrode, is spaced apart from the RV-tip electrode 112, which is a pacing electrode for the right ventricle. The first right ventricular lead system includes endocardial pacing leads that are advanced through the superior vena cava (SVC), the right atrium 120 and into the right ventricle 118 to contact myocardial tissue at a first pacing site within the right ventricle 118.

The second right ventricular lead system 115 includes an RV-tip electrode 132 and an RV-ring electrode 134. The first right ventricular lead system 104 includes endocardial pacing leads that are advanced through the superior vena cava (SVC), the right atrium 120 and into the right ventricle 118 to contact myocardial tissue at a second pacing site within the right ventricle 118.

The right atrial lead system 105 includes a RA-tip electrode 156 and an RA-ring electrode 154. The RA-tip 156 and RA-ring 154 electrodes may provide respectively pacing pulses to the right atrium of the heart and detect cardiac signals from the right atrium. In one configuration, the right atrial lead system 105 is configured as a J-lead.

In this configuration, the intracardiac lead system 102 is shown positioned within the heart 101, with the first and the second right ventricular lead systems 104, 115 extending through the right atrium 120 and into the right ventricle 118. In particular, the RV-tip electrode 112 and RV-coil electrode 114 are positioned at appropriate locations to sense and pace a first site within the right ventricle 118. The SVC-coil 116 is positioned at an appropriate location within the right atrium chamber 120 of the heart 101 or a major vein leading to the right atrium chamber 120 of the heart 101. The RV-coil 114 and SVC-coil 116 depicted in FIG. 7 are defibrillation electrodes. An RV-tip electrode 132, and an RV-ring electrode 134 are positioned at appropriate locations to sense and pace a second site within the right ventricle 118.

Referring now to FIG. 8 of the drawings, there is shown an embodiment of a reconfigurable cardiac device that incorporates CFM capabilities. The reconfigurable cardiac device, in its cardiac therapy operating mode, includes a housing 103 electrically and physically coupled to an intracardiac lead system 102 using a header 100. The intracardiac lead system 102 is implanted in a human body with portions of the intracardiac lead system 102 inserted into a heart 101. The intracardiac lead system 102 is used to detect and analyze electric cardiac signals produced by the heart 101 and to provide electrical energy to the heart 101 under certain predetermined conditions to treat cardiac arrhythmias.

The intracardiac lead system 102 includes one or more electrodes used for pacing, sensing, or defibrillation. In the particular embodiment shown in FIG. 8, the intracardiac lead system 102 includes a right ventricular lead system 104, a right atrial lead system 105, and a left atrial/ventricular lead system 106. In one embodiment, the right ventricular lead system 104 is configured as an integrated bipolar pace/shock lead.

The right ventricular lead system 104 includes an SVC-coil 116, an RV-coil 114, and an RV-tip electrode 112. The RV-coil 114, which may alternatively be configured as an RV-ring electrode, is spaced apart from the RV-tip electrode 112, which is a pacing electrode for the right ventricle.

The right atrial lead system 105 includes a RA-tip electrode 156 and an RA-ring electrode 154. The RA-tip 156 and RA-ring 154 electrodes may provide pacing pulses to the right atrium of the heart and detect cardiac signals from the right atrium. In one configuration, the right atrial lead system 105 is configured as a J-lead.

In this configuration, the intracardiac lead system 102 is shown positioned within the heart 101, with the right ventricular lead system 104 extending through the right atrium 120 and into the right ventricle 118. In particular, the RV-tip electrode 112 and RV-coil electrode 114 are positioned at appropriate locations within the right ventricle 118. The SVC-coil 116 is positioned at an appropriate location within the right atrium chamber 120 of the heart 101 or a major vein leading to the right atrium chamber 120 of the heart 101. The RV-coil 114 and SVC-coil 116 depicted in FIG. 8 are defibrillation electrodes.

An LV-tip electrode 113, and an LV-ring electrode 117 are inserted through the coronary venous system and positioned adjacent to the left ventricle 124 of the heart 101. The LV-ring electrode 117 is spaced apart from the LV-tip electrode 113, which is a pacing electrode for the left ventricle. Both the LV-tip 113 and LV-ring 117 electrodes may also be used for sensing the left ventricle, thereby providing two sensing sites within the left ventricle. The left atrial/left ventricular lead system 106 further includes two LA-ring electrodes, LA-ring1 136 LA-ring2 134, positioned adjacent the left atrium 122 for pacing and sensing the left atrium 122 of the heart 101.

The left atrial/left ventricular lead system 106 includes endocardial pacing leads that are advanced through the superior vena cava (SVC), the right atrium 120, the valve of the coronary sinus, and the coronary sinus 150 to locate the LA-ring1 136, LA-ring2 134, LV-tip 113 and LV-ring 117 electrodes at appropriate locations adjacent to the left atrium and ventricle 122,124, respectively.

According to one lead delivery approach, left atrial/ventricular lead placement involves creating an opening in a percutaneous access vessel, such as the left subclavian or left cephalic vein. The left atrial/left ventricular lead 106 is guided into the right atrium 120 of the heart via the superior vena cava. From the right atrium 120, the left atrial/left ventricular lead system 106 is deployed into the coronary sinus ostium, the opening of the coronary sinus 150. The lead system 106 is guided through the coronary sinus 150 to a coronary vein of the left ventricle 124. This vein is used as an access pathway for leads to reach the surfaces of the left atrium 122 and the left ventricle 124 which are not directly accessible from the right side of the heart.

Lead placement for the left atrial/left ventricular lead system 106 may be achieved via the subclavian vein access and a preformed guiding catheter for insertion of the LV and LA electrodes 113, 117, 136, 134 adjacent the left ventricle 124 and left atrium 122, respectively. In one configuration, the left atrial/left ventricular lead system 106 is implemented as a single-pass lead.

The components, functionality, and structural configurations depicted in FIGS. 1-8 are intended to provide an understanding of various features and combination of features that may be incorporated in a cardiac device in accordance with the present invention. It is understood that a wide variety of cardiac devices in accordance with the present invention are contemplated, ranging from relatively sophisticated to relatively simple designs. As such, particular cardiac devices in accordance with the present invention may include particular features as described herein, while other such device configurations may exclude particular features described herein.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

1. An implantable cardiac device, comprising:
an implantable housing;
a first electrode coupled to the housing and a second electrode;
monitoring circuitry coupled to the first and second electrodes, the first and second electrodes configured for cardiac activity sensing when the device is operated in a monitoring mode;
energy delivery circuitry coupled to the first and second electrodes, the first and second electrodes configured for cardiac activity sensing and energy delivery when the device is operated in an energy delivery mode;
a lead interface coupled to the housing, the lead interface configured to receive a cardiac lead; and
a controller coupled to the lead interface, monitoring circuitry, and energy delivery circuitry, the controller transitioning operation of the device from the monitoring mode to the energy delivery mode at least in part in response to coupling the cardiac lead to the lead interface.

2. The device of claim 1, further comprising detection circuitry provided in the housing and coupled to the first and second electrodes, the detection circuitry configured to receive the cardiac signals.

3. The device of claim 2, further comprising memory provided in the housing and coupled to the detection circuitry, the memory configured to store selected cardiac signals.

4. The device of claim 2, further comprising a programmable filter coupled to the detection circuitry, the programmable filter configurable in a first filtering mode for monitoring associated with the monitoring mode and configurable in a second filtering mode for cardiac event detection associated with the energy delivery mode.

5. The device of claim 1, further comprising a mode switch coupled to the controller, the mode switch configured to transition the cardiac device between the monitoring mode and the energy delivery mode.

6. The device of claim 1, further comprising a transceiver that receives a transmit request signal and transmits the contents of the memory to a patient-external device in response to receipt of the transmit request signal.

7. The device of claim 1, further comprising a receiver coupled to the controller, the controller switching the cardiac device between the monitoring mode and the energy delivery mode in response to the receiver receiving a switch request signal.

8. An implantable cardiac device, comprising:
an implantable housing;
a lead system coupled to the housing;
a first electrode coupled to the lead system, and a second electrode;
detection circuitry provided in the housing and coupled to the first and second electrodes, the detection circuitry configured to receive the cardiac signals sensed by the first and second electrodes;
memory provided in the housing and coupled to the detection circuitry, the memory configured to store selected cardiac signals;
therapy circuitry provided in the housing and coupled to the detection circuitry, the therapy circuitry configured to provide a cardiac stimulation therapy; and
a controller that transitions the cardiac device between a first operating mode and a second operating mode, the first operating mode associated with loop recording cardiac activity and non-enablement of the therapy circuitry, and the second operating mode associated with enablement of the therapy circuitry.

9. The device of claim 8, wherein the second electrode is provided in or on the housing.

10. The device of claim 8, wherein the first and second electrodes are provided in or on the housing, the second electrode electrically isolated from the first electrode.

11. The device of claim 8, further comprising a transmitter that transmits the contents of the memory to a patient-external device.

12. The device of claim 8, further comprising a transceiver that receives a transmit request signal and transmits the contents of the memory to a patient-external device in response to receipt of the transmit request signal.

13. The device of claim 8, further comprising a receiver configured to receive a switch request signal, the controller switching the cardiac device between the first operating mode and the second operating mode in response to the receiver receiving the switch request signal.

14. The device of claim 8, further comprising a programmable filter coupled to the detection circuitry, the programmable filter configurable in a first filtering mode and a second filtering mode.

15. The device of claim 14, further comprising a receiver configured to receive a program signal, the controller configuring the filter from the first filtering mode to the second filtering mode in response to receipt of the program signal.

16. The device of claim 8, wherein the controller comprises a hardware switch in or on the housing.

17. The device of claim 8, wherein the controller comprises a software switch configured to switch the cardiac device between the first operating mode and the second operating mode.

18. The device of claim 8, wherein the detection circuitry is configured to select signals associated with cardiac arrhythmic events as the selected cardiac signals for storage.

19. The device of claim 8, further comprising a lead coupled to the therapy circuitry.

20. The device of claim 19, wherein the lead comprises a pacing lead.

21. The device of claim 19, wherein the lead comprises a defibrillation or cardioversion lead.

22. The device of claim 19, wherein the lead is configured to support bi-ventricular pacing therapy.

23. An implantable cardiac device, comprising:
an implantable housing;
a header coupled to the housing;
a controller in the housing;
a first electrode and a second electrode, the first and second electrodes adapted to at least sense cardiac signals;
detection circuitry provided in the housing and coupled to the controller and the first and second electrodes, the detection circuitry configured to receive the cardiac signals;
memory provided in-the housing and coupled to the controller, the memory configured to store selected cardiac signals;
therapy circuitry provided in the housing and coupled to the controller and the first and second electrodes, the therapy circuitry configured to provide a cardiac therapy; and
an actuatable mode switch that transitions the cardiac device between a first operating mode and a second operating mode, the first operating mode disabling the therapy circuitry and associated with cardiac activity monitoring and the second operating mode enabling the therapy circuitry and associated with cardiac therapy delivery.

24. The device of claim 23, wherein the first electrode is located in or on the housing and the second electrode is coupled to the housing using the header.

25. The device of claim 23, wherein the first and the second electrode are coupled to the housing using the header.

26. The device of claim 23, wherein the header is configured to connect a cardiac lead to the therapy circuitry and to couple the second electrode to the housing.

27. The device of claim 23, wherein the header is configured to connect a cardiac lead to the therapy circuitry and to couple the first and second electrodes to the detection circuitry.

28. The device of claim 23, wherein the header is configured to connect a cardiac lead to the therapy circuitry.

29. The device of claim 28, wherein the cardiac lead comprises a defibrillation or cardioversion lead.

30. The device of claim 28, wherein the cardiac lead comprises a pacing lead.

31. The device of claim 28, wherein the cardiac lead comprises a first and second portion, the first and second portions configured to provide resynchronization pacing therapy.

32. The device of claim 28, wherein the cardiac lead comprises a memory, the memory comprising a code that actuates the mode switch.

33. The device of claim 23, wherein the mode switch is provided in or on the header, the header configured to connect a cardiac lead to the therapy circuitry, wherein connecting the therapy lead actuates switching the cardiac device between the first operating mode and the second operating mode.

34. The device of claim 23, wherein the mode switch comprises a hardware switch.

35. The device of claim 23, wherein the mode switch comprises a software switch.

36. The device of claim 23, further comprising a transmitter configured to transmit contents of the memory to a patient-external device.

37. The device of claim 23, further comprising a transceiver configured to receive a transmit request signal and transmit the contents of the memory to a patient-external device in response to receipt of the transmit request signal.

38. A cardiac system, comprising:
an implantable cardiac device configured to operate in a first operating mode and a second operating mode, the first operating mode associated exclusively with cardiac activity monitoring and the second operating mode associated with cardiac monitoring and therapy delivery, the implantable cardiac device comprising:
a housing;
a first electrode and a second electrode coupled to the housing, and configured to sense cardiac signals;
detection circuitry and energy delivery circuitry respectively provided in the housing;
a transceiver; and
a controller provided in the housing and coupled to the transceiver and the detection and energy delivery circuitry, the controller configured to store the cardiac signals in a memory and to transmit the cardiac signals in response to a transmit request signal; and
a patient-external device configured to send the transmit request signal to the transceiver.

39. The system of claim 38, wherein the patient-external device comprises a patient actuatable trigger, the trigger initiating the transmit request signal upon actuation of the trigger.

40. The system of claim 38, wherein the patient-external device comprises a patient actuatable trigger, the trigger initiating a storage request signal to the controller upon actuation of the trigger, the controller storing the cardiac signals in the memory in response to receipt of the storage request signal.

41. The system of claim 38, further comprising a programmable filter coupled to the first and second electrodes and to the transceiver, the programmable filter configured to filter the cardiac signals, wherein the transceiver is further configured to receive a program signal and program the filter from a first filter configuration to a second filter configuration in response to the program signal.

42. A cardiac monitoring and stimulation method, comprising:
providing an implantable cardiac device;
operating the cardiac device in a first mode as a loop recorder for monitoring cardiac activity and storing selected cardiac events;
enabling the implantable cardiac device to operate in a second mode as a cardiac rhythm management system; and
operating the cardiac device in the second mode to monitor cardiac activity and provide cardiac stimulation therapy once enabled.

43. The method of claim 42, further comprising selecting cardiac events for storing via patient request.

44. The method of claim 42, further comprising continuously recording cardiac events when operating in the first mode, wherein storing selected cardiac events comprises storing the most recent loop recording.

45. The method of claim 42, further comprising connecting a lead to the cardiac device, wherein switching the cardiac device between the first cardiac monitoring mode and the second mode is disabled until the lead is connected to the cardiac device.

46. The method of claim 42, wherein the cardiac stimulation therapy comprises a defibrillation or cardioversion therapy.

47. The method of claim 42, wherein the cardiac stimulation therapy comprises an antitachycardia pacing therapy.

48. The method of claim 42, wherein the cardiac stimulation therapy comprises a resynchronization pacing therapy.

49. A method, comprising:
providing an implantable cardiac device;
monitoring cardiac activity of a patient using the cardiac device;
storing cardiac event data in a memory of the cardiac device;
diagnosing, using the stored cardiac event data, the patient as having a condition requiring use of a cardiac stimulation device; and
configuring the cardiac device to operate as the cardiac stimulation device.

50. The procedure of claim 49, wherein configuring the cardiac device comprises switching the device between a first operating mode associated with cardiac activity monitoring and a second operating mode associated with cardiac therapy delivery.

51. The procedure of claim 50, wherein the switching comprises changing a hardware switch from a first position to a second position.

52. The procedure of claim 50, wherein the diagnosis is performed at least in part by use of the implanted cardiac device.

53. The procedure of claim 50, further comprising transmitting the stored cardiac event data to a patient-external device.

54. The procedure of claim 50, wherein the switching comprises updating a software program.

55. The procedure of claim 49, further comprising implanting an endocardial lead in the patient and connecting the endocardial lead to the cardiac device.

56. The procedure of claim 49, further comprising implanting an epicardial lead in the patient and connecting the epicardial lead to the cardiac device.

57. The procedure of claim 49, further comprising implanting a subcutaneous lead in the patient and connecting the subcutaneous lead to the cardiac device.

58. A cardiac device, comprising:
an implantable housing, the implantable housing comprising:
means for implantably detecting cardiac electrograms;
means for monitoring the detected electrograms;
means for transmitting the recorded electrograms to a patient-external device;
means for providing cardiac stimulation; and
means for switching the cardiac device between a monitoring mode and a cardiac stimulation mode.

59. The device of claim 58, wherein the means for switching comprises a hardware switch.

60. The device of claim 58, wherein the means for switching comprises a software switch.

61. The device of claim 58, wherein the means for switching comprises a proximity switch.

62. The device of claim 58, wherein the means for recording comprises a patient actuation means for actuating the recording means.
